(19) United States
(12) Patent Application Publication
(10) Pub. No.: US 2022/0326239 A1
(43) Pub. Date: Oct. 13, 2022

(54) METHOD FOR THE DETECTION OF PNEUMOCOCCAL INFECTION

(71) Applicants: UNIVERSITE JEAN MONNET SAINT ETIENNE, SAINT ETIENNE (FR); CENTRE HOSPITALIER UNIVERSITAIRE DE SAINT ETIENNE, SAINT ETIENNE (FR); BIOSPEEDIA, PARIS (FR)

(72) Inventors: Bruno POZZETTO, SAINT-HEAND (FR); Evelyne BEGAUD, Paris (FR); Yves GERMANI, Paris (FR); Cyrille HADDAR, Paris (FR); Anne CARRICAJO, SAINT ETIENNE (FR)

(21) Appl. No.: 17/610,764

(22) PCT Filed: May 14, 2020
(86) PCT No.: PCT/FR2020/050802

§ 371 Date: Nov. 15, 2021

(30) Foreign Application Priority Data
May 16, 2019 (FR) FR1905115

(51) Int. Cl. G01N 33/569; C12Q 1/14; G01N 33/543

(52) U.S. Cl. CPC G01N 33/56944; C12Q 1/14; G01N 33/54388

(57) ABSTRACT

The present invention relates to a method for diagnosing in vitro a pneumococcal infection (Streptococcus pneumoniae), comprising at least two steps of detecting a pneumococcal antigen: (i) on a sample of secretions from the airways of a patient, and (ii) on the same sample after dilution.

### TECHNICAL FIELD

Pneumococcal infections are caused by a bacterium called Streptococcus pneumoniae.

Streptococcus pneumoniae (pneumococcus) is a Gram-positive and catalase-negative bacterium that can be seen in isolation, in diplococci or in short chains on direct microscopic examination. The distinctive feature of the pneumococcus is that it has a capsule, one of its main virulence factors. The bacterial wall of pneumococci is composed of polysaccharides comprising carbohydrate subunits that are targets of the immune response, due to their antigenic properties.

In humans, the pneumococcus is a commensal of the respiratory tracts, i.e., it can be present in the mouth, nose and pharynx without causing pathologies: 5 to 25% of the population is an asymptomatic carrier of the bacteria.

However, pneumococci can be responsible for infections in many areas of the body: the middle ear (otitis) in children, the sinuses (sinusitis) in adults, the brain (meningitis), the blood (bacteremia) and the respiratory tract (rhinopharyngitis, bronchitis, pneumonia).

Pneumococcus can be transmitted from person to person through direct, close contact with the infected or carrier person, in particular through kissing, coughing, sneezing or hand-to-hand contact.

Some pneumococcal infections can be serious, with septicemic dissemination of the bacteria, which requires urgent and sometimes extensive treatment (venous antibiotic therapy, hospitalization or even resuscitation), with a significant mortality rate in frail or elderly subjects.

Pneumococcal infections are a major cause of morbidity and mortality worldwide. Worldwide, more than 800 000 children under the age of five die each year from pneumococcal disease, according to the World Health Organization (WHO). In France, pneumococci are the leading cause of community-acquired bacterial pneumonia and bacterial meningitis in adults. Pneumococcal infections commonly complicate viral infections, particularly influenza, especially in frail people. In young children, it is a common cause of otitis and pneumonia.

In children in particular, pneumococcus is commonly present as an asymptomatic carrier but can also be responsible for invasive infections of varying severity, some of which have a very sudden and life-threatening course. This is why it is essential to make an early distinction between an asymptomatic carriage and an early or progressive invasive infection.

### PRIOR ART

The clinical signs, the symptoms, and physical examinations alone cannot be used to distinguish S. pneumoniae pneumonia from pneumopathies due to other pathogens (other pyogenic bacteria, so-called atypical bacteria with intracellular multiplication, viruses, etc.).

The first-line biological diagnosis of S. pneumoniae pneumonia is based upon:
cytobacteriological examination of sputum (ECBC, in France), of limited interest because of the associated commensal flora, usually followed by culture on suitable medium (such as blood agar) for distinguishing and isolating the different bacterial colonies;
bronchoalveolar lavage (BAL) or pleural fluid puncture, which are highly invasive examinations but which provide a definitive diagnosis; indeed, in this type of sample, the presence of any germ indicates an invasive infection;
pneumococcal antigenuria: a simple and rapid test that detects in the urine a specific antigen common to all pathogenic pneumococcal serotypes; the antigen found in the urine comes from the degradation of the bacteria by the body's defense systems.

However, in urine, this antigen persists for several weeks and does not disappear following appropriate antibiotic therapy, which limits its diagnostic value in terms of the current infectious episode. Moreover, this diagnostic value is very variable according to studies, with a good specificity, in the order of 92 to 99% (false positives described in particular in patients with asthma or chronic obstructive pulmonary disease) and a variable sensitivity according to the clinical severity (between 66 and 82%).

A method for detecting pneumococcal antigens in secretions from a patient's respiratory tract, based on the counterimmunoelectrophoresis (CIE) technique, was first proposed in 1978 (Miller et al., 1978). However, this detection technique lacks the sensitivity for use in diagnostics.

Subsequently, kits for detecting pneumococcal antigens in respiratory tract secretions were developed, and their diagnostic efficacy (sensitivity and specificity) was compared with kits for detecting pneumococcal antigens in urine, as described in (Fukushima et al., 2015).

In particular, mention may be made the ODK0501 kit which comprises polyclonal antibodies against S. pneumoniae polysaccharide C. This test has been deemed more clinically useful than urine antigen detection kits (Ehara et al., 2008), (Izumikawa et al., 2009).

Another kit called RAPIRUN was tested in comparison with the BinaxNOW® kit used on urine samples: the RAPIRUN sputum test showed better sensitivity than the urine test (Ikegame et al., 2016).

However, in children under 12 years of age, the use of urine or sputum antigen tests appears irrelevant to the diagnosis of S. pneumoniae infection because of the high prevalence of asymptomatic pneumococcal carriage in this age group. This is especially true for children under 5 years of age, for whom the prevalence of asymptomatic carriage is estimated to be between 57 and 65%.

As of today, semi-quantitative culture on fresh blood agar and 48-hour cooked blood agar is considered the “gold standard” for the diagnosis of S. pneumoniae pneumonia, regardless of the patient's age. Thus, in the event of suspected pneumonia in children, the standard approach in the medical biology laboratory is as follows:

a) a bacterial culture is prepared on fresh blood agar and cooked blood agar,
b) optionally, depending on the clinical symptoms and the epidemiological context, a DNA gene amplification test is performed for germs responsible for atypical pneumonia, such as Mycoplasma pneumoniae or Chlamydia pneumoniae,
c) a quantitative gene amplification test for S. pneumoniae is performed, in particular by polymerase chain reaction (PCR). The most commonly used targets of PCR tests are the main autolysin gene (lytA), the pneumolysin gene (ply) and the capsular gene (cpsA). Such PCR tests for the pneumolysin gene are described in (Greiner et al., 2001) and (Saukkoriipi et al., 2004).

The disadvantages of these detection methods are as follows:

In the case of culture on agar medium, it is often difficult to detect colonies due to the polymicrobial context of the sample. Indeed, S. pneumoniae is a fragile germ, tricky to culture; since the colonies are often small, it is common not to locate them among the other colonies of less demanding, more invasive bacteria during culture.

For the gene amplification method, the primary disadvantage is that the result is positive both in the case of a true infection and in the case of an asymptomatic carriage. This technique, unless it is quantitative, is therefore unsuitable for the diagnosis of an invasive infection.

Yet, to distinguish an invasive infection from an asymptomatic carriage, it is essential to quantify or semi-quantify the bacterial population: indeed, according to the recommendations of the French Society of Microbiology (REMIC, 6^{th} edition, 2018), a high bacterial load (≥10⁷ CFU/mL) in sputum is predictive of a genuine pneumococcal infection.

Currently, apart from semi-quantitative bacterial culture, which is time-consuming and tedious, or quantitative gene amplification, which is not yet routinely available, there is no method for quantifying the bacterial population of S. pneumoniae, and thus differentiating between asymptomatic carriage and invasive infection.

### SUMMARY OF THE INVENTION

The present invention relates to a process for in vitro diagnosis of pneumococcal (Streptococcus pneumoniae) infection comprising at least two steps of detecting a pneumococcal antigen:

(i) on a sample of secretions from a patient's respiratory tract, and
(ii) on the same sample after dilution.

These biological samples from the respiratory tract (sputum, etc.) are easily obtained without invasive (traumatic) procedures for the patient.

Since the diagnostic process according to the invention is semi-quantitative, it can differentiate between an asymptomatic carriage of S. pneumoniae, common in the nasopharyngeal sphere in children (bacterial load <10⁷ CFU/mL), and the true infection (bacterial load 10⁷ CFU/mL) requiring antibiotic treatment.

The present invention also relates to a diagnostic kit for carrying out the in vitro process as described above, comprising:
an immunological test comprising at least one antibody specifically binding to at least one pneumococcal antigen; and
a dilution solution.

### BRIEF DESCRIPTION OF THE DRAWINGS

FIG. 1 shows an algorithm for the management of pneumopathy according to the diagnostic process of the invention. The PneumoResp immunological test is used for a first detection step on a pure sample (PURE) and then for a second detection step on a dilute sample (DILUTE). Diagnostic conclusions and suggested additional analyses are presented.

FIG. 2 is a correlation between the bacterial load expressed in number of PCR cycles and the quantification in culture expressed in CFU/mL (corresponding to CFU/mL) of S. pneumoniae, as a function of the amplified gene: ply or lytA. Cycle threshold (Ct) corresponds to the number of cycles at which a signal is detected in quantitative PCR. The number of cycles required to detect the bacteria in PCR is thus correlated to an effective quantity of the bacterial population.

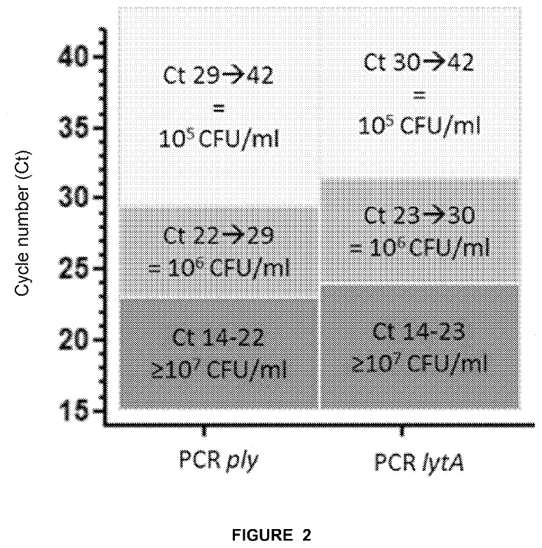

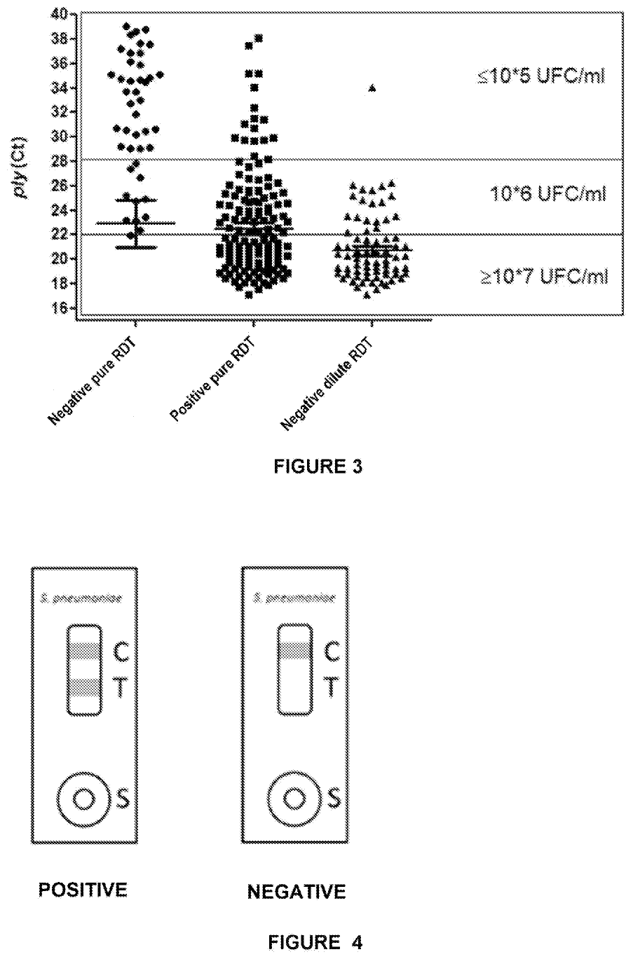

FIG. 3 shows a box plot of the correlation between the amount of S. pneumoniae (CFU/mL) as determined by the number of PCR cycles for ply gene amplification (based on the results of FIG. 2), and the results of the detection steps of the process according to the invention:
a negative pure rapid diagnostic test (RDT) represents the samples for which a negative result was obtained in the first detection step;
a positive pure RDT represents the samples for which a positive result was obtained in the first detection step;
a positive diluted RDT represents the samples for which a positive result was obtained in the second detection step.

The large horizontal bars represent the median for each situation. The small lower and upper bars represent the 25% and 75% quartile, respectively.

FIG. 4 shows a reading of the positive/negative results of the PneumoResp test. The line C is the internal control which should be positive, the line T is the sample test. The circle S represents the well in which a few drops of sample and migration buffer were placed to perform the test.

### DETAILED DESCRIPTION

The present invention relates to a process for in vitro diagnosis of pneumococcal (also referred to as Streptococcus pneumoniae or S. pneumoniae) infection comprising at least two steps of detecting a pneumococcal antigen:

(i) on a sample of secretions from a patient's respiratory tract, and
(ii) on the same sample after dilution.

“Pneumococcal infection” is understood to mean an invasive (as opposed to colonizing) infection, also known as a “true” infection, characterized by the presence of a significant amount of S. pneumoniae bacteria in a sample.

The present application particularly relates to pneumococcal respiratory infections, characterized by the presence of a significant number of bacteria of the species S. pneumoniae in a sample of respiratory secretions (nasopharyngeal secretions, sputum, tracheal secretions, etc.) of human origin. According to the recommendations of the French Society of Microbiology (REMIC, 6th edition, 2018), a high bacterial load 10⁷ CFU/mL) in a sputum respiratory sample is predictive of invasive pneumococcal infection.

Invasive respiratory pneumococcal infection includes S. pneumoniae pneumonia, but also higher respiratory infections (bronchitis, rhinopharyngitis, and even sinusitis or acute otitis media)

The present invention also relates to a process for in vitro diagnosis of pneumococcal infection comprising a step of detecting a pneumococcal antigen on a sample of secretions from a patient's respiratory tract, said sample being diluted.

“Sample after dilution” or “diluted sample” is understood to mean a sample of secretions from a patient's respiratory tract to which a certain volume of a suitable solution has been added in order to evaluate the concentration of bacteria present in the sample. In so doing, the results obtained from the immunological test are more accurate and reliable. The dilution factor will be chosen by persons skilled in the art on the basis of their general knowledge. The dilution factor should be adapted to the sensitivity threshold of the immunological test used and to the patient population tested.

For example, the dilution factor is comprised between 2 and 1000, or between 10 and 500, or between 20 and 200, or between 50 and 150, or between 10 and 100, the bounds of each interval being included in the interval.

In a preferred embodiment, the dilution factor is 100.

The “result” or “information” obtained by the process, or by one of the steps of the process, is understood to mean a figure representative of the presence and quantity of pneumococci in the tested sample. This “result” gives an indication to persons skilled in the art who can conclude on the state of infection of a patient, on the basis of this figure or these data.

This procedure for in vitro diagnosis of pneumococcal infection is semi-quantitative, allowing patients with “true infection” to be distinguished from those with asymptomatic carriage.

The advantages of these diagnostic procedures according to the invention are in particular the following:
These procedures can be carried out within a time comprised between 15 and 40 minutes, and in any case less than one hour; they are very simple to implement and require very few reagents and no special equipment; this very short time (less than one hour from the start of the processing of the sample) to obtain information is compatible with making a therapeutic decision within a time that optimizes the chances of survival of the patient tested;
Obtaining the sample of secretions from an individual's respiratory tract is easy, using a non-invasive procedure;
A negative result at the first detection step, on an undiluted sample, makes it possible to exclude with near certainty a serious pneumococcal infection, which avoids:
   a) for the laboratory, an unnecessary search for this germ,
   b) for the patient and the microbial ecology, inappropriate antibiotic therapy (the search can be focused on other bacterial or viral pathogens),
   c) for the community, unnecessary or prolonged hospitalization.

This type of diagnostic procedure is also commonly referred to as a rapid diagnostic test (RDT) or rapid diagnostic orientation test (RDOT). It is a so-called “first-line” test that allows the practitioner to determine whether other detection procedures must be implemented to confirm or invalidate the initial conclusions, or whether the information obtained by implementing the process according to the invention is sufficient to engage in a particular therapeutic approach.

### Nature of the Sample and Prior Fluidization

The expression “secretions from a patient's respiratory tract” is understood to mean all secretions from the upper and lower respiratory tracts of the human body, excluding saliva.

In particular, this includes any upper respiratory tract sample, nasopharyngeal secretions, tracheobronchial secretions (sputum), induced sputum, nasopharyngeal aspirate, bronchial aspirate, and bronchoalveolar lavage (BAL) fluid.

According to a preferred embodiment, the sample of secretions from a patient's respiratory tract is fluidized prior to performing the diagnostic procedure.

This fluidization can be achieved by any means known to the skilled person, such as mechanical treatment of the sample (ultrasonic treatment, shaking in the presence of microbeads, etc.) or chemical treatment with a fluidizing agent.

Several chemical products having a capacity of fluidizing a secretion are known to the skilled person. For example, the fluidizing agent may be dithiothreitol or N-acetyl cysteine diluted to 50%. In particular, the fluidizing agent may be selected from the following commercial products Digest-EU R® (Eurobio), Sputasol (ThermoFisher) or Mucomyst® (EurekaSanté).

Conventionally, one volume of fluidizing agent is added for one volume of sputum sample (dilution by a factor of 2). However, it is understood that the skilled person will be able to adapt the volume of fluidizing agent used, depending on the nature and consistency of the sample.

### Patient Population

Any individual susceptible to pneumococcal infection may be tested according to the in vitro diagnostic process of the invention.

In the present application, the terms “individual” and “patient” are used interchangeably, and both refer to the human being from whom the respiratory tract secretions used in the diagnostic process according to the invention are obtained.

As mentioned above, this diagnostic procedure is particularly suitable for the subpopulation of children under 12 years of age, and preferably for children under 5 years of age.

Thus, according to one embodiment of the process, the patient from whom the respiratory tract secretions are derived is less than 12 years of age or is less than 5 years of age.

However, the diagnostic process according to the invention may also be applied to patients over 12 years of age, in particular to adults, and especially to persons over 70 years of age.

### Immunological Test Used for the Detection Steps

The in vitro diagnostic process is characterized in that the detection steps are performed by a single immunological test comprising at least one antibody specifically binding to at least one pneumococcal antigen.

Said immunological test is characterized by (i) the nature of the antibody or antibodies comprised in said test and (ii) the type of immunological test (reagents, visualization of results). These two characteristics are expanded upon below.

### Antibody/Antigen Pair

The immunological test comprises an antibody specifically recognizing at least one pneumococcal antigen.

The concept of “specific antibody/antigen binding” is understood to mean, in the sense of the invention, that the antibody recognizes and captures an antigen of S. pneumoniae in a specific manner, i.e., without cross-reacting with antigens of other bacterial species.

Advantageously, the immunological test used will not cross-react with any of the following bacterial species potentially present in the respiratory flora: Alloscardovia omnicolens, Enterococcus avium, Enterococcus faecalis, Enterococcus faecium, Haemophilus influenzae, Haemophilus parainfluenzae, Klebsiella pneumoniae, Staphylococcus aureus, Streptococcus agalactiae, Streptococcus anginosus, Streptococcus constellatus, Streptococcus gordonii, Streptococcus pyogenes, Streptococcus salivarius, Streptococcus sanguinis, Streptococcus parasanguinis, Streptococcus mitis and Streptococcus vestibularis.

The S. pneumoniae antigen detected may be of any molecular nature, including a protein, lipid or sugar, allowing specific identification of S. pneumoniae in a sample containing other bacterial species.

Each detection step may detect at least one pneumococcal antigen, i.e., one or more pneumococcal-specific antigens, and this with a single antibody or with several antibodies.

According to an advantageous embodiment, the immunological test used is compatible with the main chemical fluidizers used in hospital medical biology laboratories.

Indeed, most fluidizers generate an oxidation of the thiol groups of the molecules when they are added to a biological sample. Thus, the structure of certain antigens can be modified, in particular that of protein antigens, which is likely to alter the recognition of said modified antigen by an antibody.

Consequently, when the biological sample has been previously fluidized by the addition of a chemical fluidizer, it is advantageous that the antibody or antibodies are directed against an antigen not modified by the fluidizer, for example an antigen of a non-protein nature, in particular an antigen consisting of a lipid or a sugar.

According to a preferred embodiment, the antibody comprised in the immunological test is capable of specifically detecting at least one polysaccharide antigen of S. pneumoniae.

According to a particular embodiment, the antibody or antibodies comprised in said immunological test is/are directed against one or more polysaccharides of S. pneumoniae.

### Immunological Test Used for a Step of Detecting at Least One Pneumococcal Antigen

In the sense of the invention, “immunological test” is understood to mean a test for detecting at least one S. pneumoniae antigen by means of at least one antibody specifically recognizing this antigen, said antibody being coupled to a detectable reagent, or to an enzyme reacting to the addition of a detection reagent.

Said detection reagent may in particular be a colored, fluorescent, luminescent reagent, or any type of detection reagent that can be detected and/or quantified by techniques well known to the skilled person.

The skilled person knows many types of immunological tests for detecting specific antigens. The present diagnostic procedure can be performed with any type of immunological test which specifically identifies at least one S. pneumoniae antigen.

A commonly used immunological test is, for example, the enzyme-linked immunosorbent assay (ELISA), in which two types of antibodies are used, one being specific to the antigen to be detected, and the other reacting to antigen-antibody complexes and being coupled to an enzyme capable of generating the emission of a signal in the presence of a chromogenic or fluorogenic substrate.

Other conventional immunological tests are referred to as “immunochromatographic”. These tests combine antigenic detection by antibody with chromatographic membrane migration of the antigen/antibody complex.

Some immunochromatographic tests are based on the use of antibodies coupled to nanoparticles, particularly gold nanoparticles. The migration of the antibodies, possibly bound to at least one antigen, takes place on a suitable membrane. The results can be read quickly, usually within 10 to 20 minutes. The immunological test should be used according to the instructions given in the test package insert.

According to a preferred embodiment of the process according to the invention, the immunological test used to detect one or more S. pneumoniae antigen(s) is an immunochromatographic test. In other words, according to a particular embodiment, the process of the invention is characterized in that each detection step is performed by means of an immunochromatographic test.

According to a particular embodiment, the immunological test is an immunochromatographic test comprising at least one antibody specifically binding to an S. pneumoniae polysaccharide.

According to another particular embodiment, the immunological test is an immunochromatographic test comprising at least one antibody specifically binding to S. pneumoniae polysaccharide C.

While most commercially available S. pneumoniae-specific immunochromatographic tests are indicated for detecting pneumococci in urine or cerebrospinal fluid samples, these tests can also be used, in the context of the process according to the invention, on samples from a patient's respiratory tract.

Thus, the tests that can be used for implementing the diagnostic process according to the invention are in particular the following:
the PneumoSpeed test marketed by BIOSPEEDIA,
the BinaxNOW® test marketed by ALERE,
the Tru Strep Pneumo™ test marketed by MERIDIAN,
the Uni-Gold™ S. pneumoniae test marketed by TRINITY BIOTECH,
the Sofia S. pneumoniae FIA test marketed by QUIDEL,
the Pneumo test marketed by BIOSYNEX.

Other tests developed specifically for detecting respiratory tract secretions may be used, such as the Rapirun® and ODK0501 kits.

Advantageously, the detection steps of the process according to the invention implement the PneumoResp S. pneumoniae polysaccharide detection test developed by the company BIOSPEEDIA, the characteristics of which are presented in Example 4.

### Process for Semi-Quantification of Pneumococci Present in the Sample

According to a particular embodiment of the process according to the invention, steps (i) and (ii) are carried out successively, i.e., the process comprises at least two steps of detecting a pneumococcal antigen:

(i) on a sample of secretions from a patient's respiratory tract, then
(ii) on the same sample after dilution by a suitable factor, for example by a dilution factor of 10 to 1000, preferentially by a factor of 100.

The diagnostic process according to the invention is characterized in that at the end of each detection step a “positive” or “negative” result is obtained by carrying out the immunological test.

Advantageously, this result is a semi-quantitative result, as it allows the quantity of pneumococci present in the sample to be evaluated.

A “negative” result obtained after the first detection step (i) is representative of a quantity of pneumococci in the sample that is less than or equal to 10³ CFU/mL. This quantity was determined by comparing the results obtained on more than 200 samples subjected to several tests in parallel:

a) quantitative bacteriological culture,
b) PCR amplification of the S. pneumoniae DNA, and
c) the process according to the invention.

Such a negative result may be interpreted as representative of the absence of invasive pneumococcal infection and will lead to the conclusion that the patient tested does not have invasive pneumococcal infection.

Of the 65 patient samples tested according to the process of the invention, showing a negative result following the first detection step on an undiluted sample, no S. pneumoniae pneumopathy was clinically diagnosed.

In certain cases, said sample has been pre-fluidized, by adding a certain volume of a fluidizing agent. Because of this prior dilution, it would not be correct to refer to the process of the invention as “quantitative”; nevertheless, the term “semi-quantitative” process is applicable here insofar as reliable information on the quantity of pneumococci present in the sample is obtained by the implementation of the detection test, which has been compared with the results obtained by conventional semi-quantification methods, as presented in the examples of the application.

In the experiments presented in the experimental section, after diluting the sample 1:100, the sensitivity of the test is close to the threshold of 10⁷ CFU/mL (see FIG. 3), which corresponds to the value considered significant for recognizing the involvement of this bacterium in the etiology of a pulmonary parenchymal infection.

Skilled persons may carry out as many dilutions of the sample and detection steps as they wish: for example, the process may comprise a detection step on a pure sample, a detection step on a sample diluted by a factor of 10, then a detection step on a sample diluted by a factor of 100.

The sample will be diluted in a suitable solution.

The dilution factor will be chosen by persons skilled in the art based on their general knowledge. Preferably, the dilution factor will be adapted to the sensitivity threshold of the immunological test used and/or to the patient population tested.

For example, the dilution factor is comprised between 2 and 1000, or between 10 and 500, or between 20 and 200, or between 50 and 150, or between 10 and 100, the bounds of each interval being included in the interval.

According to a preferred embodiment, the dilution factor is 100, i.e., a dilution of a factor of 10⁻².

According to an embodiment of the process, the process further comprises a step (iii) of comparing the results obtained in steps (i) and (ii).

Indeed, the results obtained in the detection steps (i) and (ii) may be either similar (positive then positive) or different (positive then negative).

Depending on the agreement or disagreement of the results, information on the quantity of pneumococci present in the respiratory tract secretion sample can be obtained.

Indeed, and as presented in the experimental part, a correlation between the quantitative results of the bacteriological culture and the results of the immunological test used in the process of the invention could be put in parallel, and thus make it possible to affirm that:
if both detection steps (i) and (ii) resulted in a positive result, this is representative of a quantity of pneumococci in the sample greater than or equal to 10⁷ CFU/mL; and
if step (i) resulted in a positive result and step (ii) resulted in a negative result, this is representative of a quantity of pneumococci in the sample comprised between about 10³ and about 10⁶ CFU/mL.

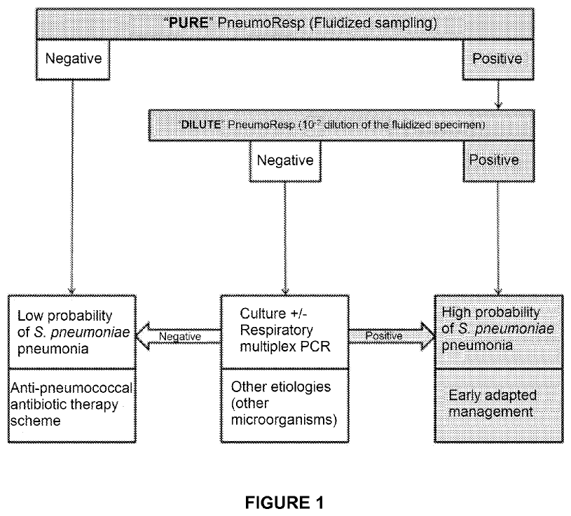

Thus, as shown in FIG. 1, if both detection steps (i) and (ii) have resulted in a “positive” result, then the patient is likely to have an invasive pneumococcal infection.

Conversely, if detection step (i) resulted in a “positive” result and detection step (ii) resulted in a “negative” result, then the patient is likely an asymptomatic carrier of pneumococcus.

Naturally, other complementary diagnostic methods may be performed to confirm these presumptions.

Advantageously, the diagnostic process according to the invention makes it possible to distinguish between the following two cases:
patients with an invasive pneumococcal infection, and
patients who are asymptomatic carriers of S. pneumoniae or are following a recent but ended infection.

According to an embodiment of the invention, the in vitro diagnostic process comprises the following steps:

i) detecting the presence of at least one pneumococcal antigen, on a sample of secretions from a patient's respiratory tract, by implementing an immunological test;
ii) diluting said sample, then detecting the presence of at least one pneumococcal antigen by implementing the same immunological test as in (i) on said diluted sample;
iii) comparing the results obtained in steps (i) and (ii).

This process is shown schematically in FIG. 1.

According to a particular embodiment, the diagnostic process consists of the three steps (i), (ii) and (iii).

According to another embodiment, the diagnostic process comprises other additional steps, and in particular:
a step of bacteriological culture on agar of the tested sample, to confirm the absence of an invasive pneumococcal infection; and/or
a step of amplification of an S. pneumoniae gene in the tested sample;
these additional steps being recommended in particular in the case of different results between the first and second detection steps.

According to an embodiment of the process, the process further comprises a step (iv) of bacteriological culture from the sample of secretions from the patient's respiratory tract.

The present application also relates to a process for treating an invasive pneumococcal infection, comprising the following steps:

i) detecting the presence of at least one pneumococcal antigen in a sample of secretions from the respiratory tract of an individual likely to be affected by an invasive pneumococcal infection, by carrying out an immunological test;
ii) diluting said sample, then detecting the presence of at least one pneumococcal antigen by implementing the same immunological test as in (i) on said diluted sample;
iii) comparing the results obtained in steps (i) and (ii),
iv) in the event that both detection steps (i) and (ii) lead to a positive result, administering an antibiotic suitable for said individual.

The suitable antibiotic will be one with specific action against S. pneumoniae, or a broad spectrum antibiotic with action against S. pneumoniae.

### Diagnostic Kit

The present invention also relates to a diagnostic kit for carrying out the in vitro process as described above, comprising:
an immunological test comprising at least one antibody specifically binding to at least one pneumococcal antigen; and
a dilution solution suitable for diluting a sample of secretions from an individual's respiratory tract.

Advantageously, this kit will comprise reagents suitable for the pneumococcal antigen detection step.

Advantageously, said kit will also comprise an instruction manual.

Advantageously, said kit can be stored at room temperature.

According to another embodiment, the kit will further comprise a fluidizing agent to be added to the secretion sample.

A kit comprising a fluidizing agent will preferably be stored at a temperature of less than 5° C., for the purpose of storing the fluidizing agent in liquid form.

However, if the fluidizing agent is incorporated into the kit as a powder, then the kit will comprise a suitable solvent for dissolving the fluidizing agent, and the kit can be stored at room temperature.

### EXAMPLES

### Example 1. Test Population and Conventional Diagnostic Tests

### 1.1. Patient Population

One hundred and ninety-six (196) respiratory tract samples (nasopharyngeal secretions and sputum) from children admitted to the University Hospital of Saint-Etienne (France) were collected. The subjects included were less than 15 years of age and were admitted to the pediatric emergency room, pediatrics or intensive care unit. Clinical, radiological and biological data were collected and analyzed a posteriori.

The diagnosis of S. pneumoniae pneumonia was defined on the coexistence of:

(i) suggestive clinical criteria,
(ii) an abnormal parenchymal image on chest radiography, and
(iii) a culture greater than or equal to 10⁷ CFU of S. pneumoniae in semi-quantitative bacteriology. (Harris et al., 2011).

The demographic and clinical characteristics of the patient cohort are listed in Table 1 below.

TABLE 1

| Characteristics | Numerical data | |
|---|---|---|
| Demographic characteristics | | |
| Number of subjects | 196 | |
| Average age (years) | 1.99 | (0-15) |
| Median age (years) | 0.83 | |
| Interquartile range (years) | 0.18-2.88 | |
| Sex ratio (boys/girls) | 1.20 | |
| Reason for consultation | | |
| Dyspnea | 92 | (46.9%) |
| Cough | 39 | (19.9%) |
| Hyperthermia | 77 | (39.3%) |
| Other (mainly abdominal pain and diarrhea) | 36 | (18.4%) |
| Antibiotic therapy prior to emergency room | 23 | (11.7%) |
| consultation | | |
| Chest X-ray | 124 | (63.3%) |
| Lung focus | 32 | |
| Interstitial syndrome | 54 | |
| Services | | |
| Pediatric emergencies | 187 | (95.4%) |
| Resuscitation | 9 | (4.6%) |
| Average length of stay (days) | 7.8 | |
| Number of subjects hospitalized | 154 | (78.6%) |
| Biological inflammatory syndrome | 63 | (3.2%) |
| Respiratory diseases | 86 | (43.9%) |
| S. pneumoniae pneumonia | 23 | (11.7%) |

### Demographic and Clinical Characteristics of the Patient Cohort

1.2 Bacteriological Identification of S. pneumoniae by Culture

The samples collected were fluidized with an equivalent volume of a sterile dithiothreitol solution (Digest-EUR®, Eurobio, France) for semi-quantitative cultures.

The undiluted pretreated sample and 10⁻²-fold dilutions were plated on 4 agar media: blood agar (COS, BioMérieux) with an optochin disc (OPTO-F, BioMérieux), Columbia ANC agar based on dehydrated medium (BBL™ Columbia agar, BD) and defibrinated sheep blood (Thermofisher), bromocresol lactose purple agar (BCP agar, BioMérieux) and chocolate agar (PVX agar, BioMérieux). The plates were incubated in an oven at 37° C. under 5% CO₂ (Arbique et al., 2004).

A reading was taken at 24 h and 48 h and the colony count was expressed in CFU/mL.

Colonies were identified presumptively on phenotypic characteristics: flat, alpha hemolytic and optochin sensitive colonies (diameter >15 mm), then confirmed with a Microflex LT mass spectrometer (Bruker Daltonics, Bremen, Germany).

In addition, a test tube bile salt lysis test was performed on each isolated strain, following the manufacturer's recommendations (BBL™ Desoxycholate Reagent Droppers, BD). A positive result with this test discriminates the species Streptococcus pneumoniae from other non-pneumoniae streptococci, mitis group.

1.3. PCR Amplification of S. pneumoniae Genes

Two polymerase chain reaction (PCR) techniques were used to detect S. pneumoniae directly in patient samples, one targeting the ply (pneumolysin) gene and the other the lytA (autolysin) gene, which correspond to both main virulence factors of the bacteria.

Briefly, 200 μL of sample was pre-treated with 20 μL of proteinase K (Eurobio, France) for 10 minutes at 50° C. and then extracted using the EasyMag automated system (BioMérieux, France) following the supplier's recommendations. Samples were eluted in 110 μL of elution buffer and stored frozen at −20° C. before testing. The primers and probes used (Eurogentec, France) are presented in Table 2 (Carvalho et al., 2007).

TABLE 2

| | | 5′-3′ | Size | |
|---|---|---|---|---|
| Name | Type | sequences | (bp) | Reference |
| ply gene | | | | |
| -SPply F | Sense | TGCAGAGCGTC | 22 | SEQ ID |
| | | CTTTGGTCTAT | | NO: 1 |
| -SPply R | Anti- | CTCTTACTCGTG | 25 | SEQ ID |
| | sense | GTTTCCAACTTGA | | NO: 2 |
| -SPply TP | Probe | TTCGAGTGTTGC | 24 | SEQ ID |
| | | TTATGGGCGCCA | | NO: 3 |
| lytA gene | | | | |
| -LytA F | Sense | CGCAATCTAGC | 22 | SEQ ID |
| | | AGATGAAGCAG | | NO: 4 |
| -LytA R | Anti- | AAGGGTCAACG | 21 | SEQ ID |
| | sense | TGGTCTGAGTG | | NO: 5 |
| -LytA Pr | Probe | TTTGCCGAAAACG | 25 | SEQ ID |
| | | CTTGATACAGGG | | NO: 6 |

Primer and Probe Sequences Used for the Detection and Identification of S. pneumoniae

### 1.4. The Microbiological Results of the 196 Samples in the Study are Presented in Table 3 Below.

TABLE 3

| Technique | Numerical data |
|---|---|
| Culture positive for S. pneumoniae | 70 |
| <10⁷ CFU/mL | 20 |
| ≥10⁷ CFU/mL | 50 |
| Positive PCR tests (ply and/or lytA) | 169 (86.2%) |
| ply | 167 (85.2%) |
| lytA | 123 (62.7%) |

### Microbiological Results of the 196 Samples of Example 1

Semi-Quantitative Cultures for S. pneumoniae

Of the 196 samples, 70 strains of S. pneumoniae were isolated. The semi-quantitative data are shown in Table 3. These strains were all sensitive to optochin and the bile salt lysis tests were also all positive.

Quantitative PCR Tests and Correlation with Culture

Out of 196 samples, 167 were positive for the ply gene and 123 positive for the lytA gene. The sensitivity of both techniques was evaluated by determining the limit of quantification: 10² CFU/mL for ply and 10³ CFU/mL for lytA.

The correlation between the positivity cycle (Ct) of each of both PCR reactions, which is proportional to the bacterial load of the sample, and the semi-quantification of the culture of a suspension of S. pneumoniae, was performed using a colony counter (Scan 1200, Interscience). The respective Ct's of each PCR were correlated to the quantification in CFU/mL of S. pneumoniae and the equivalence results of these two semi-quantitative techniques are presented in FIG. 2.

Next, a correlation was made between the Ct of the PCR test detecting the ply gene expressed in CFU/mL taking into account the values in FIG. 2, and the results of the semi-quantitative culture (Table 4).

TABLE 4
Equivalence between semi-quantification by culture and by RT-PCR.

| Quantification | PCR ply quantification | | | |
|---|---|---|---|---|
| culture | ≥10⁷ CFU/mL | 10⁶ CFU/mL | 10⁵ CFU/mL | ≤10² CFU/mL* |
| ≥10⁷ CFU/mL | 42 | 8 | 0 | 0 |
| 10⁶ CFU/mL | 10 | 8 | 0 | 0 |
| 10⁵ CFU/mL | 0 | 2 | 0 | 0 |
| ≤10⁴ CFU/mL** | 5 | 45 | 47 | 29 |

*PCR ply detection threshold
**Threshold of quantification of the bacteriological culture

Among the 99 PCR-positive and culture-negative samples, 49 (49.5%) had a bacterial load (assessed by PCR)^{≤10}^{⁵} CFU/mL and 15 (15.2%) corresponded to patients on antibiotic treatment, which explains this discrepancy.

### Example 2. Process for Diagnosing S. pneumoniae Infection Using a PneumoResp Immunochromatographic Test

### 2.1. Steps of the Process Implemented

PneumoResp is an immunochromatography test for detecting S. pneumoniae polysaccharide antigens.

PneumoResp uses a pair of antibodies directed against polysaccharides. Gold nanoparticles coupled to the specific antibodies will interact with the polysaccharide antigens if they are present in the sample; upon migration with the sample along a nitrocellulose membrane, the complexes of polysaccharide antigens and gold-conjugated antibody bind to the test line (immunocapture line), resulting in the appearance of a reddish band at the test line T (if the test is positive) and the control line C (whether the test is positive or negative) The absence of the control band indicates an uninterpretable test.

This test is indicated in the figures as RDT, for rapid diagnostic test.

Step 1—The sputum sample is fluidized by adding (1 volume of sample to 1 volume of diluent) of a reagent such as Digest-EUR® (Eurobio) or Sputasol (ThermoFisher).
Step 2—Remove roughly 1 milliliter of fluidized sputum and place in the tube 1; vortex for 10 seconds.
Step 3—Place one drop of the processed sample from the tube 1 into the sample well S, then three drops of the PneumoResp migration buffer (Diluent Buffer). Start the timer. A reddish colored front moves upwards along the membrane. The reading is done with the naked eye (it can also be done with a suitable reader). The results are only valid after the control line has appeared. The final reading of the test is taken at the 15th minute, but positive results may appear earlier.
Step 4—If this first test is positive, the processed sample from the tube 1 is then processed in the tube 2. To this end, take 10 microliters of the treated sample remaining in the tube 1 and mix with the contents of the tube 2 (1:100 dilution). Vortex the mixture for 10 seconds.
Step 5—Step 3 is repeated.

The results of steps 3 and 5 are said to be “positive” if a red band appears in the line T and in the line C. They are said to be “negative” if no band appears in the line T but a line appears in the line C, as shown in FIG. 4.

The first detection step is called “PURE”, i.e., performed on an undiluted sample; the second detection step is called “DILUTE”, i.e., performed on a sample diluted by a factor of 100.

The specificity of the test was evaluated on 52 bacterial strains belonging to 24 different species including 30 strains belonging to the genus Streptococcus of the mitis group (Streptococcus mitis, Streptococcus oralis, Streptococcus gordonii, Streptococcus sanguinis, Streptococcus parasanguinis, Streptococcus peroris, Streptococcus pneumoniae and Streptococcus pseudopneumoniae) and 22 strains of bacteria belonging to other bacterial genera (Alloscardovia omnicolens, Enterococcus avium, Enterococcus faecalis, Enterococcus faecium, Haemophilus influenzae, Haemophilus parainfluenzae, Klebsiella pneumoniae, Parvimonas micra and Staphylococcus aureus) or streptococci not belonging to the mitis group (Streptococcus agalactiae, Streptococcus anginosus, Streptococcus constellatus, Streptococcus pyogenes, Streptococcus salivarius, and Streptococcus vestibularis)) in order to verify the absence of cross-reactions with the antigens used with respect to the bacteria potentially present in the respiratory flora

2.2. Results Obtained with the PneumoResp Test:

The test was evaluated on 196 respiratory tract samples (nasopharyngeal secretions and sputum) from children admitted to the University Hospital of Saint-Etienne, France. The subjects included were less than 15 years of age and were admitted to the pediatric emergency room, pediatrics or intensive care unit.

Of the 196 samples, 63 were negative and 133 positive in the first detection step. The samples found positive were retested after dilution. At the end of this second detection step, 76 samples were positive and 57 negative.

### Example 3. Comparison of the Results Obtained in Example 1 with Conventional Diagnostic Protocols (Bacteriological Culture, PCR) and the Results Obtained by Implementing the Process According to the Invention in Example 2

### 3.1. Statistical Analysis

Descriptive variables, sensitivity (Se), specificity (Sp), and positive (PPV) and negative (NPV) predictive values were reported with their 95% confidence intervals (CI). Parametric and non-parametric tests and graphs were performed using the GraphPad Prism 5 software (California, USA). P values less than 5% were considered statistically significant.

### 3.2 Performance Comparison: Raw Results

The performance of the diagnostic process according to the invention, compared with that of conventional techniques (bacteriological culture and gene amplification by PCR) is presented in Table 5 below.

TABLE 5
Comparison of the performance of the diagnostic process according to the invention with the
results of conventional techniques (bacteriological culture and PCR gene amplification)

| | Culture positive for S. pneumoniae | | | | | | | |
|---|---|---|---|---|---|---|---|---|
| RDT | | | Sensitivity | | Specificity | VPP | VPN | |
| result | Positive | Negative | (95% CI) | | (95% CI) | (95% CI) | (95% CI) | |
| Total | | | | | | | | |
| (n = 196) | | | | | | | | |
| Pure RDT | | | 100 | (94.8-100) | 50.4 (41.4-58.6) | 52.6 (48.3-56.9) | 100 | (94.3-100) |
| Positive | 70 | 63 | | | | | | |
| Negative | 0 | 63 | | | | | | |
| Diluted | | | 91.4 | (82.5-96.0) | 80.9 (69.6-88.7) | 84.2 (76.1-89.9) | 89.5 | (79.8-94.9) |
| RDT | | | | | | | | |
| Positive | 64 | 12 | | | | | | |
| Negative | 6 | 51 | | | | | | |

| | Culture ≥10⁷ CFU/mL of S. pneumoniae | | | | | |
|---|---|---|---|---|---|---|
| | | | Sensitivity | Specificity | VPP | VPN |
| | Positive | Negative | (95% CI) | (95% CI) | (95% CI) | (95% CI) |
| Total | | | | | | |
| (n = 196) | | | | | | |
| Pure RDT | | | 100 (92.9-100) | 43.1 (35.4-51.2) | 37.6 (34.3-41.0) | 100 (94.3-100) |
| Positive | 50 | 83 | | | | |
| Negative | 0 | 63 | | | | |
| Diluted | | | 94.6 (92.9-100) | 68.7 (58.1-77.4) | 65.8 (58.3-72.6) | 100 (93.7-100) |
| RDT | | | | | | |
| Positive | 50 | 26 | | | | |
| Negative | 0 | 57 | | | | |

| | Quantitative PCR ≥10⁷ CFU/mL (ply gene) | | | | | |
|---|---|---|---|---|---|---|
| | | | Sensitivity | Specificity | VPP | VPN |
| | Positive | Negative | (95% CI) | (95% CI) | (95% CI) | (95% CI) |
| Total | | | | | | |
| (n = 196) | | | | | | |
| Pure RDT | | | 98.4 (91.5-99.7) | 46.6 (38.3-55.1) | 46.6 (42.7-50.7) | 98.4 (89.8-99.8) |
| Positive | 62 | 71 | | | | |
| Negative | 1 | 62 | | | | |
| Diluted | | | 91.9 (82.5-96.5) | 73.2 (61.9-82.1) | 75.0 (67.0-81.6) | 91.2 (82.0-96.0) |
| RDT | | | | | | |
| Positive | 57 | 19 | | | | |
| Negative | 5 | 52 | | | | |

| Quantitative PCR ≥10⁷ CFU/mL (lytA gene) | | | | | |
|---|---|---|---|---|---|
| | | Sensitivity | Specificity | VPP | VPN |
| Positive | Negative | (95% CI) | (95% CI) | (95% CI) | (95% CI) |

| | | | | | | | |
|---|---|---|---|---|---|---|---|
| Total | | | | | | | |
| (n = 196) | | | | | | | |
| Pure RDT | | | 100 (87.5-100) | 37.3 (30.3-44.8) | 20.3 (18.5-22.3) | 100 | (94.3-100) |
| Positive | 27 | 106 | | | | | |
| Negative | 0 | 63 | | | | | |
| Diluted | | | 96.3 (81.7-99.3) | 52.8 (43.4-62.1) | 34.2 (29.3-39.2) | 98.3 | (89.0-99.7) |
| RDT | | | | | | | |
| Positive | 26 | 50 | | | | | |
| Negative | 1 | 56 | | | | | |

| | S. pneumoniae pneumonia | | | | | |
|---|---|---|---|---|---|---|
| | | | Sensitivity | Specificity | VPP | VPN |
| | Positive | Negative | (95% CI) | (95% CI) | (95% CI) | (95% CI) |
| Total | | | | | | |
| (n = 196) | | | | | | |
| Pure RDT | | | 100 (85.7-100) | 36.4 (29.6-43.8) | 17.3 (15.7-19.0) | 100 (94.3-100) |
| Positive | 23 | 110 | | | | |
| Negative | 0 | 63 | | | | |
| Diluted | | | 100 (85.7-100) | 51.8 (42.6-60.9) | 30.3 (26.3-34.5) | 100 (93.7-100) |
| RDT | | | | | | |
| Positive | 23 | 53 | | | | |
| Negative | 0 | 57 | | | | |

### 3.3. Performance of the Process According to the Invention in Relation to the Results of Bacterial Culture:

The performance of this test compared with S. pneumoniae culture was as follows:
sensitivity (Se) of 100% [95% CI: 94.8-100],
specificity (Sp) of 50.4% [95% CI: 41.4-58.6],
positive predictive value (PPV) of 52.6% [95% CI: 48.3-56.9] and
negative predictive value (NPV) of 100% [95% CI: 94.3-100].

The Se and NPV remain 100% against S. pneumoniae culture ≥10⁷ CFU/mL (100% [95% CI: 92.9-100]) and against lytA PCR with a quantification threshold ≥10⁷ CFU/mL (100% [95% CI: 87.5-100]).

The Se for ply PCR was 98.4% [95% CI: 91.5-99.7]. Indeed, only one sample was found negative at the first detection step, and had a Ct close to the threshold (Ct of 21.96 for 22 cycles) for ply PCR (see FIG. 3).

Moreover, out of the 196 samples, only one was positive in the first detection step, and at the same time negative in ply PCR.

Of the 63 negative tests in the first detection step, no child had S. pneumoniae pneumonia. The NPV and Se were 100%.

3.4. A Process Comprising Two Detection Steps, on a Pure Sample and then on a Diluted Sample:

The samples found positive were retested after dilution. At the end of this second step, 76 samples were found positive and 57 were found negative.

The performance of this process compared with culture was as follows:
Se of 91.4% [95% CI: 82.5-98.0],
Sp of 80.9% [95% CI: 69.6-88.7],
PPV of 84.2% [95% CI: 76.1-89.9], and
NPV of 89.5% [95% CI: 79.8-954.9].

Taking into account the quantified culture ≥10⁷ CFU/mL of S. pneumoniae, the performance is as follows:
Se of 94.6% [95% CI: 92.9-100],
Sp of 68.7 [95% CI: 58.1-77.4],
PPV of 65.8% [95% CI: 58.3-72.6], and
NPV of 100% [95% CI: 93.7-100].

In addition, all cases of S. pneumoniae pneumonia (n=23) were found positive in the second detection step. The performance of the test after the dilution step in relation to the clinical diagnosis of S. pneumoniae pneumonia was as follows:
Se of 100% [95% CI: 85.7-100],
Sp of 51.8% [95% CI: 42.6-60.9],
PPV of 30.3% [95% CI: 26.3-34.5], and
VPN 100% [95% CI: 93.7-100].

### 3.5. Summary Analysis of the Results of the Study

In this study, the results obtained were compared with semi-quantitative culture on fresh blood agar and 48-hour cooked blood agar, considered the “gold standard” for the diagnosis of S. pneumoniae pneumonia (Table 6).

For all patients found negative in the first detection step (n=63), clinical and radiological analysis coupled with a negative culture for pneumococcus excluded the diagnosis of pneumococcal disease. These results indicate a sensitivity of 100% (23/23) and a negative predictive value of 100% (63/63).

TABLE 6
Comparison of semi-quantitative culture and PneumoResp results on
undiluted fluidized sputum from 196 pediatric sputum samples.

| | | Semi-quantitative culture | | |
|---|---|---|---|---|
| | | Positive | Negative | Total |
| PneumoResp | Positive | 70 | 63 | 133 |
| on pure fluidized | Negative | 0 | 63 | 63 |
| sputum | Total | 70 | 126 | 196 |

This excellent negative predictive value of the procedure performed on undiluted fluidized sputum makes it possible exclude pneumococcal pneumonia with near certainty in the event of a negative test result.

The 133 positive sputum samples were retested after dilution of the respiratory secretions (detection step 2). The results were compared with the final diagnosis of pneumococcal disease based on clinical-radiological data, biological work-up, search for other respiratory pathogens and a positive pneumococcal culture at a threshold of at least 10⁷ CFU per mL (REM IC).

All patients labeled in this study as having pneumococcal disease (n=23) were found to be positive in both detection steps (undiluted and after 1:1 dilution) by the process of the invention.

The excellent sensitivity (23/23, 100%) of the process of the invention with 2 steps of detection on fluidified sputum makes it possible to suspect, from the first day and with a very high probability, an invasive pneumococcal infection and thus to provide a suitable antibiotic treatment at an early stage.

### Example 4. Characteristics of the PneumoResp Immunochromatographic Test

### Materials and Reagents Provided:

twenty (20) individual “test” cassettes contained in hermetically sealed bags with desiccant
twenty (20) single-use plastic dropper pipettes
a preparatory treatment vial “Tube 1” and compatibility of the fluidized sample and PneumoResp reagents
a sample preparation vial “Tube 2” for the second test in case of a positive result of the first test
a pusher diluent vial
a positive control vial
an instruction manual.

### Equipment and Reagents Required but not Provided:

a micropipette with disposable tips for withdrawing 10 microliters
a vortex
a micropipette with disposable tips for withdrawing 1 milliliter
a stopwatch or timer
standard sterile bottles for collection of sputum, bronchial secretions or sputum
a container for biological waste
disposable gloves
   Important: To interpret the test, it is essential to observe the presence of the control line; it ensures that the volume of sample tested is sufficient and that the procedure described is correct. This control line must appear within 15 minutes (before or after the test line if it is positive).
   Positive test: Presence of two distinct bands: a control line appears in the area C and a reddish line, even of low intensity, appears in the area T.
   Negative test: Only a reddish line appears in the control area C. No line appears in the area T.
   Invalid test: No visible reddish line appears in the control area C regardless of the result in the area T.

An internal control system is integrated in each cassette. Indeed, the change of color from pink to purple on the control line (C) is considered as an internal control. A colored line appears on the control line to indicate that the user has put in a sufficient volume of sample and that the test is running under good conditions. A strong coloration of the reading area on the cassette may cause the control to read incorrectly. It should be very light pink to white within 15 minutes and should not interfere with the reading of the results.

### LIST OF DOCUMENTS CITED

Miller J, Sande M A, Gwaltney J M Jr, Hendley J O. Diagnosis of pneumococcal pneumonia by antigen detection in sputum. J Clin Microbiol. 1978 May; 7(5):459-62.
Fukushima K, Nakamura S, Inoue Y, Higashiyama Y, Ohmichi M, Ishida T, Yoshimura K, Sawai T, Takayanagi N, Nakahama C, Kakugawa T, Izumikawa K, Aoki N, Nishioka Y, Kosaka O, Kohno S. Utility of a sputum antigen detection test in pneumococcal pneumonia and lower respiratory infectious disease in adults. Intern Med. 2015; 54:2843-50.
Izumikawa, K., Akamatsu, S., Kageyama, A., Okada, K., Kazuyama, Y., Takayanagi, N., & Ishida, T. (2009). Evaluation of a rapid immunochromatographic ODK0501 assay for detecting Streptococcus pneumoniae antigen in sputum samples from patients with lower respiratory tract infection. Clin. Vaccine Immunol., 16, 672-8.
Ehara, N., Fukushima, K., Kakeya, H., Mukae, H., Akamatsu, S., Kageyama, A.,& Kohno, S. (2008). A novel method for rapid detection of Streptococcus pneumoniae antigen in sputum and its application in adult respiratory tract infections. Journal of medical microbiology, 57, 820-6.
Ikegame, S., Nakano, T., Otsuka, J., Yoshimi, M., Matsuo, T., Kubota, M., & Takata, S. (2017). The evaluation of the sputum antigen kit in the diagnosis of pneumococcal pneumonia. Internal Medicine, 56, 1141-6.
Greiner O, Day P J, Bosshard P P, Imeri F, Altwegg M, Nadal D. (2001) Quantitative detection of Streptococcus pneumoniae in nasopharyngeal secretions by real-time PCR. J Clin Microbiol, 39:3129-34.
Saukkoriipi A, Leskelä K, Herva E, Leinonen M. (2004) Streptococcus pneumoniae in nasopharyngeal secretions of healthy children: comparison of real-time PCR and culture from STGG-transport medium. Mol Cell Probes, 18:147-53.
Harris, M., Clark, J., Coote, N., Fletcher, P., Harnden, A., McKean, M., Thomson, A. (2011). British Thoracic Society guidelines for the management of community acquired pneumonia in children: update 2011. Thorax, 66 (Suppl 2), ii1-ii23.
Arbique J C, Poyart C, Trieu-Cuot P, Quesne G, Carvalho MGS, Steigerwalt A G, Morey R E, Jackson D, Davidson R J, Facklam R R. (2004) Accuracy of phenotypic and genotypic testing for identification of Streptococcus pneumoniae and description of Streptococcus pseudopneumoniae sp. nov. J Clin Microbiol, 42:4686-96.
Carvalho Mda G, Tondella M L, McCaustland K, Weidlich L, McGee L, Mayer L W, Steigerwalt A, Whaley M, Facklam R R, Fields B, Carlone G, Ades E W, Dagan R, Sampson J S. (2007) Evaluation and improvement of real-time PCR assays targeting lytA, ply, and psaA genes for detection of pneumococcal DNA. J Clin Microbiol, 45:2460-6.

1-11. (canceled)

12. A process for in vitro diagnosis of pneumococcal (Streptococcus pneumoniae) infection comprising at least two steps of detection of a pneumococcal antigen:
(i) a first step of detection on a sample of secretions from a patient's respiratory tract, and
(ii) a second step of detection on the same sample, after its dilution.

13. The diagnostic process as claimed in claim 12, wherein the second step (ii), the sample is diluted by a dilution factor of 10 to 1000, preferentially by a factor of 100.

14. The diagnostic process of claim 12, wherein each detection step (i) and (ii) is performed by means of an immunological test comprising at least one antibody specifically binding to at least one pneumococcal antigen.

15. The diagnostic process of claim 12, wherein the sample has been previously fluidized.

16. The diagnostic process of claim 12, wherein at the end of each detection step (i) or (ii), a “positive” or “negative” result is obtained.

17. The diagnostic process of claim 16, wherein obtaining a “negative” result in detection step (i) is representative of a quantity of pneumococci in the sample less than or equal to 10^{3 CFU}/mL.

18. The diagnostic process of claim 12, further comprising a step (iii) of comparing the results obtained in steps (i) and (ii).

19. The diagnostic process of claim 18, wherein:
if both detection steps (i) and (ii) result in a positive result, this is representative of a quantity of pneumococci in the sample greater than or equal to 10⁷ CFU/mL; and
if step (i) results in a positive result and step (ii) results in a negative result, this is representative of a quantity of pneumococci in the sample comprised between about 10³ and about 10⁶ CFU/mL.

20. The diagnostic process of claim 12, wherein the patient is under 12 years of age.

21. The diagnostic process of claim 12, further comprising a step (iv) of bacteriological culture of the sample of secretions obtained from the patient's respiratory tract.

22. A diagnostic kit for carrying out the in vitro process as claimed in claim 12, comprising:
an immunological test comprising at least one antibody specifically binding to at least one pneumococcal antigen; and
a dilution solution.


SEQUENCE LISTING (6 sequences)

SEQ ID NO: 1 - DNA, 22 bp, Artificial Sequence
  Oligonuclotide
tgcagagcgtcctttggtctat

SEQ ID NO: 2 - DNA, 25 bp, Artificial Sequence
  Oligonuclotide
ctcttactcgtggtttccaacttga

SEQ ID NO: 3 - DNA, 24 bp, Artificial Sequence
  Oligonuclotide
ttcgagtgttgcttatgggcgcca

SEQ ID NO: 4 - DNA, 22 bp, Artificial Sequence
  Oligonuclotide
cgcaatctagcagatgaagcag

SEQ ID NO: 5 - DNA, 22 bp, Artificial Sequence
  Oligonuclotide
aagggtcaacgtggtctgagtg

SEQ ID NO: 6 - DNA, 25 bp, Artificial Sequence
  Oligonuclotide
tttgccgaaaacgcttgatacaggg